(19) United States
(12) Patent Application Publication
(10) Pub. No.: US 2009/0124645 A1
(43) Pub. Date: May 14, 2009

(54) Novel Pyrimidine-2,4-Diamine Derivatives and their Use as Modulators of Small-Conductance Calcium-Activated Potassium Channels

(75) Inventors: Ulrik Svane Sorensen, Soborg (DK); Birgitte L. Eriksen, Farum (DK); Lene Teuber, Vaerlose (DK); Dan Peters, Malmo (DK); Dorte Strobaek, Farum (DK); Tina Holm Johansen, Smorum (DK); Palle Christophersen, Ballerup (DK)

Correspondence Address: BIRCH STEWART KOLASCH & BIRCH, PO BOX 747, FALLS CHURCH, VA, 22040-0747, US

(73) Assignee: NeuroSearch A/S, Ballerup (DK)

(21) Appl. No.: 12/083,433

(22) PCT Filed: Oct. 13, 2006
(86) PCT No.: PCT/EP2006/067387

§ 371 Date: Jun. 24, 2008

Related U.S. Application Data
(60) Provisional application No. 60/726,508, filed on Oct. 14, 2005.

(30) Foreign Application Priority Data
Oct. 14, 2005 (DK) PA 2005 01439

(51) Int. Cl. A61K 31/505; C07D 239/24; A61P 25/18; A61P 25/28; A61P 9/10; A61P 11/06; A61P 9/06; A61P 13/10; A61P 9/00; A61P 1/00; A61P 27/02

(52) U.S. Cl. 514/272; 544/326

(57) ABSTRACT

This invention relates to novel pyrimidine-2,4-diamine derivatives useful as modulators of small-conductance calcium-activated potassium channels (SK channels). In other aspects the invention relates to the use of these compounds in a method for therapy and to pharmaceutical compositions comprising the compounds of the invention.

### TECHNICAL FIELD

This invention relates to novel pyrimidine-2,4-diamine derivatives useful as modulators of small-conductance calcium-activated potassium channels (SK channels). In other aspects the invention relates to the use of these compounds in a method for therapy and to pharmaceutical compositions comprising the compounds of the invention.

### BACKGROUND ART

Three subtypes of small-conductance calcium-activated potassium channels (SK channels) have been cloned: SK1, SK2 and SK3 (corresponding to KCNN1-3 using the genomic nomenclature). The activity of these channels is determined by the concentration of free intracellular calcium ([Ca²⁺]ᵢ) via calmodulin that is constitutively bound to the channels. SK channels are tightly regulated by [Ca²⁺]ᵢ in the physiological range being closed at [Ca²⁺]ᵢ up to around 0.1 μM but fully activated at a [Ca²⁺]ᵢ of 1 μM. Being selective for potassium, open or active SK channels have a hyperpolarizing influence on the membrane potential of the cell. SK channels are widely expressed in the central nervous system. The distribution of SK1 and SK2 show a high degree of overlap and display the highest levels of expression in neocortical, limbic and hippocampal areas in the mouse brain. In contrast, the SK3 channels show high levels of expression in the basal ganglia, thalamus and the brain stem monoaminergic neurons e.g. dorsal raphe, locus coeruleus and the ventral tegmental area (Sailer et al. “Comparative immunohistochemical distribution of three small-conductance Ca²⁺-activated potassium channel subunits, SK1, SK2 and SK3 in mouse brain, Mol. Cell. Neurosci. 2004, 26, 458-469). The SK channels are also present in several peripheral cells including skeletal muscle, gland cells, liver cells and T-lymphocytes.

The hyperpolarizing action of active SK channels plays an important role in the control of firing pattern and excitability of excitable cells. SK channel inhibitors such as apamin and bicuculline-methobromide have been demonstrated to increase excitability whereas the opener 1-EBIO is able to reduce electrical activity. In non-excitable cells where the amount of Ca²⁺ influx via voltage-independent pathways is highly sensitive to the membrane potential an activation of SK channels will increase the driving force whereas a blocker of SK channels will have a depolarising effect and thus diminish the driving force for calcium.

Based on the important role of SK channels in linking [Ca²⁺]ᵢ and membrane potential, SK channels are an interesting target for developing novel therapeutic agents.

A review of SK channels and SK channel modulators may be found in Liegeois, J.-F. et al.: “Modulation of small conductance calcium-activated potassium (SK) channels: a new challenge in medicinal chemistry”, Current Medicinal Chemistry, 2003, 10, 625-647.

Known modulators of SK channels suffer from being large molecules or peptides (apamin, scyllatoxin, tubocurarine, dequalinium chloride, UCL1684) or having low potency (1-EBIO, riluzole). Thus, there is a continued need for compounds with an optimized pharmacological profile. In particular, there is a great need for selective ligands, such as SK3 channel modulators.

### SUMMARY OF THE INVENTION

In its first aspect, the invention provides pyrimidine-2,4-diamine derivatives of Formula I:

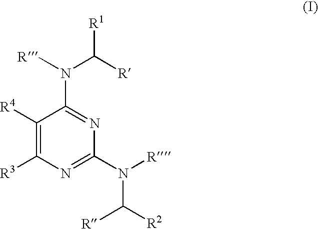

including any isomers or any mixture of isomers, and pharmaceutically acceptable salts thereof, wherein R¹, R², R³, R⁴, R′, R″ R′″ and R″″ are as defined below.

In its second aspect, the invention provides a pharmaceutical composition, comprising a therapeutically effective amount of the pyrimidine-2,4-diamine derivatives of the invention, including any isomers or any mixture of isomers, and pharmaceutically acceptable salts thereof, together with at least one pharmaceutically acceptable carrier, excipient or diluent.

In a further aspect, the invention provides the use of the pyrimidine-2,4-diamine derivatives of the invention, including any isomers or any mixture of isomers, and pharmaceutically acceptable salts thereof, for the manufacture of a pharmaceutical composition for the treatment, prevention or alleviation of a disease or a disorder or a condition of a mammal, including a human, which disease, disorder or condition is responsive to modulation of SK channels.

In a still further aspect, the invention relates to a method for treatment, prevention or alleviation of a disease or a disorder or a condition of a living animal body, including a human, which disorder, disease or condition is responsive to modulation of SK channels, which method comprises the step of administering to such a living animal body in need thereof a therapeutically effective amount of the pyrimidine-2,4-diamine derivatives of the invention, including any isomers or any mixture of isomers, and pharmaceutically acceptable salts thereof.

Other objects of the invention will be apparent to the person skilled in the art from the following detailed description and examples.

### DETAILED DISCLOSURE OF THE INVENTION

### Pyrimidine-2,4-diamine Derivatives

In its first aspect the present invention provides a pyrimidine-2,4-diamine derivative of Formula I:

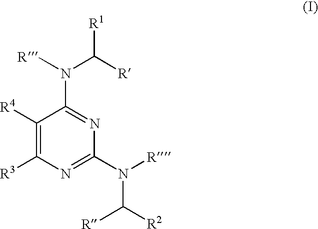

any of its isomers or any mixture of its isomers, or a pharmaceutically acceptable salt thereof; wherein

R¹ represents —(CH₂)ᵥ—R⁵; wherein v is 0 or 1; and R⁵ represents an aryl group, which aryl group is optionally substituted with one or more substituents independently selected from the group consisting of halo, trifluoromethyl, trifluoromethoxy, cyano, alkyl, amino or N,N-dialkyl-amino; and

R′ and R′″, independent of each other, represent hydrogen or R^{e}-alkyl; or R′ together with R′″ form —(CH₂)ₚ—, wherein p is 3, 4 or 5; or R′ forms a —(CH₂)_{q}— bridge to an ortho position of the aryl group of R¹, wherein q is 2, 3 or 4; and R′″ represents hydrogen or R^{e}-alkyl; wherein R^{e} represents hydrogen, hydroxyl, cyano, amino or N,N-dialkyl-amino; and

R² represents —(CH₂)_{w}—R⁶; wherein w is 0 or 1; and R⁶ represents an aryl group, which aryl group is optionally substituted with one or more substituents independently selected from the group consisting of halo, trifluoromethyl, trifluoromethoxy, cyano, alkyl, amino or N,N-dialkyl-amino; and

R″ and R″″ independent of each other represent hydrogen or R^{f}-alkyl; or R″ together with R″″ form —(CH₂)ₛ—, wherein s is 3, 4 or 5; or R″ forms a —(CH₂)ₜ— bridge to an ortho position of the aryl group of R², wherein t is 2, 3 or 4; and R″″ represents hydrogen or R^{f}-alkyl; wherein R^{f} represents hydrogen, hydroxyl, cyano, amino or N,N-dialkyl-amino; and

R³ and R⁴ independent of each other are selected from the group consisting of hydrogen, halo, trifluoromethyl, trifluoromethoxy, cyano, alkyl and alkoxy;

provided, however, that the compound is not
N²,N⁴-Bis(dibenzyl)pyrimidine-2,4-diamine;
N²,N⁴-Bis(dibenzyl)-5-fluoropyrimidine-2,4-diamine;
N²,N⁴-Bis(dibenzyl)-6-fluoropyrimidine-2,4-diamine;
N²,N⁴-Bis(dibenzyl)-6-chloropyrimidine-2,4-diamine;
N²,N⁴-Bis(dibenzyl)-6-methylpyrimidine-2,4-diamine; or
N²,N⁴-Bis(1-phenylethyl)-6-chloropyrimidine-2,4-diamine.

In a preferred embodiment the pyrimidine-2,4-diamine derivative of the invention is a compound of Formula I, wherein R¹ represents —(CH₂)ᵥ—R⁵; wherein v is 0 or 1; and R⁵ represents an aryl group, which aryl group is optionally substituted with one or more substituents independently selected from the group consisting of halo, trifluoromethyl, trifluoromethoxy, cyano, alkyl, amino or N,N-dialkyl-amino.

In a more preferred embodiment R¹ represents a phenyl group, optionally substituted with one or more substituents independently selected from the group consisting of halo, trifluoromethyl, trifluoromethoxy, cyano, alkyl, amino or N,N-dialkyl-amino.

In an even more preferred embodiment R¹ represents a phenyl group substituted with one or two times with substituents independently selected from the group consisting of halo, trifluoromethyl, trifluoromethoxy, cyano, alkyl, amino or N,N-dialkyl-amino.

In a still more preferred embodiment R¹ represents a phenyl group substituted with one or two times with substituents independently selected from the group consisting of halo, trifluoromethyl and N,N-dimethyl-amino.

In another more preferred embodiment R¹ represents a benzyl group, optionally substituted with one or more substituents independently selected from the group consisting of halo, trifluoromethyl, trifluoromethoxy, cyano and alkyl.

In an even more preferred embodiment R¹ represents a benzyl group, optionally substituted with halo, trifluoromethyl, trifluoromethoxy, cyano or alkyl.

In a still more preferred embodiment R¹ represents a benzyl group, optionally substituted with halo.

In another preferred embodiment the pyrimidine-2,4-diamine derivative of the invention is a compound of Formula I, wherein R′ and R′″, independent of each other, represent hydrogen or R^{e}-alkyl; or R′ together with R′″ form —(CH₂)ₚ—, wherein p is 3, 4 or 5; or R′ forms a —(CH₂)_{q}— bridge to an ortho position of the aryl group of R¹, wherein q is 2, 3 or 4; and R′″ represents hydrogen or R^{e}-alkyl; wherein R^{e} represents hydrogen, hydroxyl, cyano, amino or N,N-dialkyl-amino.

In a more preferred embodiment R′ and R′″, independent of each other, represent hydrogen or R^{e}-alkyl; and wherein R^{e} represents hydrogen, hydroxyl, cyano, amino or N,N-dialkyl-amino.

In an even more preferred embodiment R′ and R′″, independent of each other, represent hydrogen, alkyl or N,N-dialkyl-amino.

In a still more preferred embodiment R′ and R′″, independent of each other, represent hydrogen, methyl or N,N-dimethyl-amino.

In a third preferred embodiment the pyrimidine-2,4-diamine derivative of the invention is a compound of Formula I, wherein R² represents —(CH₂)_{w}—R⁶; wherein w is 0 or 1; and R⁶ represents an aryl group, which aryl group is optionally substituted with one or more substituents independently selected from the group consisting of halo, trifluoromethyl, trifluoromethoxy, cyano, alkyl, amino or N,N-dialkyl-amino.

In a more preferred embodiment R² represents a phenyl group, which phenyl group is optionally substituted with one or more substituents independently selected from the group consisting of halo, trifluoromethyl, trifluoromethoxy, cyano, alkyl, amino or N,N-dialkyl-amino.

In an even more preferred embodiment R² represents a phenyl group, which phenyl group is optionally substituted with one or two substituents independently selected from the group consisting of halo, trifluoromethyl, trifluoromethoxy, cyano, alkyl, amino or N,N-dialkyl-amino.

In a still more preferred embodiment R² represents a phenyl group, which phenyl group is optionally substituted with one or two substituents independently selected from the group consisting of halo, trifluoromethyl or N,N-dimethyl-amino.

In another more preferred embodiment R² represents a benzyl group, which aryl group is optionally substituted with one or more substituents independently selected from the group consisting of halo, trifluoromethyl, trifluoromethoxy, cyano and alkyl.

In an even more preferred embodiment R² represents a benzyl group, which aryl group is optionally substituted with halo, trifluoromethyl, trifluoromethoxy, cyano or alkyl.

In a still more preferred embodiment R² represents a benzyl group, which aryl group is optionally substituted with halo.

In a fourth preferred embodiment the pyrimidine-2,4-diamine derivative of the invention is a compound of Formula I, wherein R″ and R″″ independent of each other represent hydrogen or R^{f}-alkyl; or R″ together with R″″ form —(CH₂)ₛ—, wherein s is 3, 4 or 5; or R″ forms a —(CH₂)ₜ— bridge to an ortho position of the aryl group of R², wherein t is 2, 3 or 4; and R″″ represents hydrogen or R^{f}-alkyl; wherein R^{f} represents hydrogen, hydroxyl, cyano, amino or N,N-dialkyl-amino.

In a more preferred embodiment R″ and R″″ independent of each other represent hydrogen or R^{f}-alkyl; wherein R^{f} represents hydrogen, hydroxyl, cyano, amino or N,N-dialkyl-amino.

In an even more preferred embodiment R″ and R″″ independent of each other, represent hydrogen, alkyl hydroxyl-alkyl or N,N-dialkyl-amino.

In a still more preferred embodiment R″ represents hydrogen or alkyl and R″″ represents hydrogen, alkyl hydroxyl-alkyl or N,N-dialkyl-amino.

In a yet more preferred embodiment R″ represents hydrogen or methyl; and R″″ represents hydrogen, methyl, hydroxyl-methyl, hydroxyl-ethyl or N,N-dimethyl-amino.

In a fifth preferred embodiment the pyrimidine-2,4-diamine derivative of the invention is a compound of Formula I, wherein R³ and R⁴ independent of each other are selected from the group consisting of hydrogen, halo, trifluoromethyl, trifluoromethoxy, cyano, alkyl and alkoxy.

In a more preferred embodiment R³ and R⁴ independent of each other are selected from the group consisting of hydrogen and alkyl.

In an even more preferred embodiment R³ represents hydrogen or alkyl; and R⁴ represents hydrogen.

In a still more preferred embodiment R³ represents hydrogen or methyl; and R⁴ represents hydrogen.

In another more preferred embodiment R³ and R⁴ both represent hydrogen.

In a sixth preferred embodiment the pyrimidine-2,4-diamine derivative of the invention is a compound of Formula I, wherein

R′, R″, R′″ and R″″ independent of each other are hydrogen or alkyl;

R¹ represents an aryl group; which aryl group is optionally substituted with one or more substituents independently selected from the group consisting of halo, trifluoromethyl, trifluoromethoxy, cyano and alkyl;

R² represents an aryl group; which aryl group is optionally substituted with one or more substituents independently selected from the group consisting of halo, trifluoromethyl, trifluoromethoxy, cyano and alkyl; and

R³ and R⁴ independent of each other are selected from the group consisting of hydrogen, halo, trifluoromethyl, trifluoromethoxy, cyano, alkyl and alkoxy.

In a seventh preferred embodiment the pyrimidine-2,4-diamine derivative of the invention is a compound of Formula I, wherein R¹ represents

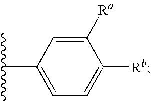

wherein R^{a} and R^{b} independent of each other are selected from the group consisting of hydrogen, halo, trifluoromethyl, trifluoromethoxy, cyano and alkyl.

In an eight preferred embodiment the pyrimidine-2,4-diamine derivative of the invention is a compound of Formula I, wherein

R² represents

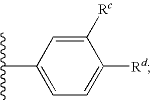

wherein R^{c} and R^{d} independent of each other are selected from the group consisting of hydrogen, halo, trifluoromethyl, trifluoromethoxy, cyano and alkyl.

In a ninth preferred embodiment the pyrimidine-2,4-diamine derivative of the invention is a compound of Formula I, wherein R³ represents hydrogen or alkyl.

In a tenth preferred embodiment the pyrimidine-2,4-diamine derivative of the invention is a compound of Formula I, wherein R⁴ represents hydrogen or alkyl.

In an eleventh preferred embodiment the pyrimidine-2,4-diamine derivative of the invention is a compound of Formula I, wherein R′ represents hydrogen or alkyl.

In a twelfth preferred embodiment the pyrimidine-2,4-diamine derivative of the invention is a compound of Formula I, wherein R″ represents hydrogen or alkyl.

In a thirteenth preferred embodiment the pyrimidine-2,4-diamine derivative of the invention is a compound of Formula I, wherein R′″ and R″″ represent hydrogen.

In a most preferred embodiment the pyrimidine-2,4-diamine derivative of the invention is
N²,N⁴-Bis(3,4-difluorobenzyl)pyrimidine-2,4-diamine;
N²,N⁴-Bis(3,4-difluorobenzyl)-6-methylpyrimidine-2,4-diamine;
N²,N⁴-Bis[3-(trifluoromethyl)benzyl]pyrimidine-2,4-diamine;
N²,N⁴-Bis(3,4-dichlorobenzyl)pyrimidine-2,4-diamine;
N²,N⁴-Bis[1-(4-fluorophenyl)ethyl]pyrimidine-2,4-diamine;
N²,N⁴-Bis[4-fluoro-3-(trifluoromethyl)benzyl]pyrimidine-2,4-diamine;
N²,N⁴-Bis(4-chlorobenzyl)pyrimidine-2,4-diamine;
N²,N⁴-Bis(4-chlorobenzyl)-N²,N⁴-dimethyl-pyrimidine-2,4-diamine;
2-{Benzyl[4-(4-chlorobenzylamino)pyrimidin-2-yl]amino}ethanol;
N²,N⁴-Bis-[2-(4-fluoro-phenyl)-ethyl]-pyrimidine-2,4-diamine;
N²,N⁴-Bis-[2-(4-chloro-phenyl)-ethyl]-pyrimidine-2,4-diamine;
N²,N⁴-Di-(R)-1,2,3,4-tetrahydronaphthalen-1-yl-pyrimidine-2,4-diamine;
N²,N⁴-Dibenzyl-N²,N⁴-bis(2-dimethylaminoethyl)pyrimidine-2,4-diamine; or
N²,N⁴-Bis(4-dimethylaminobenzyl)pyrimidine-2,4-diamine;

or a pharmaceutically acceptable salt thereof.

Any combination of two or more of the embodiments as described above is considered within the scope of the present invention.

### Definition of Substituents

In the context of this invention halo represents fluoro, chloro, bromo or iodo.

In the context of this invention an alkyl group designates a univalent saturated, straight or branched hydrocarbon chain. The hydrocarbon chain preferably contains of from one to six carbon atoms (C₁₋₆-alkyl), including pentyl, isopentyl, neopentyl, tertiary pentyl, hexyl and isohexyl. In a preferred embodiment alkyl represents a C₁₋₄-alkyl group, including butyl, isobutyl, secondary butyl, and tertiary butyl. In another preferred embodiment of this invention alkyl represents a C₁₋₃-alkyl group, which may in particular be methyl, ethyl, propyl or isopropyl.

Alkoxy is O-alkyl, wherein alkyl is as defined above.

In the context of this invention an aryl group designates a carbocyclic aromatic ring system such as phenyl, naphthyl (1-naphthyl or 2-naphthyl) or fluorenyl.

A preferred aryl group of the invention is phenyl.

### Pharmaceutically Acceptable Salts

The pyrimidine-2,4-diamine derivative of the invention may be provided in any form suitable for the intended administration. Suitable forms include pharmaceutically (i.e. physiologically) acceptable salts, and pre- or prodrug forms of the pyrimidine-2,4-diamine derivative of the invention.

Examples of pharmaceutically acceptable addition salts include, without limitation, the non-toxic inorganic and organic acid addition salts such as the hydrochloride, the hydrobromide, the nitrate, the perchlorate, the phosphate, the sulphate, the formate, the acetate, the aconate, the ascorbate, the benzenesulphonate, the benzoate, the cinnamate, the citrate, the embonate, the enantate, the fumarate, the glutamate, the glycolate, the lactate, the maleate, the malonate, the mandelate, the methanesulphonate, the naphthalene-2-sulphonate, the phthalate, the salicylate, the sorbate, the stearate, the succinate, the tartrate, the toluene-p-sulphonate, and the like. Such salts may be formed by procedures well known and described in the art.

Examples of pharmaceutically acceptable cationic salts of a pyrimidine-2,4-diamine derivative of the invention include, without limitation, the sodium, the potassium, the calcium, the magnesium, the zinc, the aluminium, the lithium, the choline, the lysinium, and the ammonium salt, and the like, of a pyrimidine-2,4-diamine derivative of the invention containing an anionic group. Such cationic salts may be formed by procedures well known and described in the art.

In the context of this invention the “onium salts” of N-containing compounds are also contemplated as pharmaceutically acceptable salts. Preferred “onium salts” include the alkyl-onium salts, the cycloalkyl-onium salts, and the cycloalkylalkyl-onium salts.

Examples of pre- or prodrug forms of the pyrimidine-2,4-diamine derivative of the invention include examples of suitable prodrugs of the substances according to the invention include compounds modified at one or more reactive or derivatizable groups of the parent compound. Of particular interest are compounds modified at a carboxyl group, a hydroxyl group, or an amino group. Examples of suitable derivatives are esters or amides.

The pyrimidine-2,4-diamine derivative of the invention may be provided in dissoluble or indissoluble forms together with a pharmaceutically acceptable solvent such as water, ethanol, and the like. Dissoluble forms may also include hydrated forms such as the monohydrate, the dihydrate, the hemihydrate, the trihydrate, the tetrahydrate, and the like. In general, the dissoluble forms are considered equivalent to indissoluble forms for the purposes of this invention.

### Steric Isomers

It will be appreciated by those skilled in the art that the pyrimidine-2,4-diamine derivatives of the present invention may contain one or more chiral centers, and that such compounds exist in the form of isomers.

Moreover, the pyrimidine-2,4-diamine derivative of the present invention may exist as enantiomers in (+) and (−) forms as well as in racemic forms (+). The racemates of these isomers and the individual isomers themselves are within the scope of the present invention.

The invention includes all such isomers and any mixtures thereof including racemic mixtures.

Racemic forms can be resolved into the optical antipodes by known methods and techniques. One way of separating the isomeric salts is by use of an optically active acid, and liberating the optically active amine compound by treatment with a base. Another method for resolving racemates into the optical antipodes is based upon chromatography on an optical active matrix. Racemic compounds of the present invention can thus be resolved into their optical antipodes, e.g., by fractional crystallisation of d- or l- (tartrates, mandelates, or camphorsulphonate) salts for example.

The pyrimidine-2,4-diamine derivative of the present invention may also be resolved by the formation of diastereomeric amides by reaction of the chemical compounds of the present invention with an optically active activated carboxylic acid such as that derived from (+) or (−) phenylalanine, (+) or (−) phenylglycine, (+) or (−) camphanic acid or by the formation of diastereomeric carbamates by reaction of the chemical compound of the present invention with an optically active chloroformate or the like.

Additional methods for the resolving the optical isomers are known in the art. Such methods include those described by Jaques J, Collet A, & Wilen S in “Enantiomers, Racemates, and Resolutions”, John Wiley and Sons, New York (1981).

Optical active compounds can also be prepared from optical active starting materials.

### Labelled Compounds

The pyrimidine-2,4-diamine derivative of the invention may be used in their labelled or unlabelled form. In the context of this invention the labelled compound has one or more atoms replaced by an atom having an atomic mass or mass number different from the atomic mass or mass number usually found in nature. The labelling will allow easy quantitative detection of said compound.

The labelled compounds of the invention may be useful as diagnostic tools, radio tracers, or monitoring agents in various diagnostic methods, and for in vivo receptor imaging.

The labelled isomer of the invention preferably contains at least one radionuclide as a label. Positron emitting radionuclides are all candidates for usage. In the context of this invention the radionuclide is preferably selected from ²H (deuterium), ³H (tritium), ¹³C, ¹⁴C, ¹³¹I, ¹²⁵I, ¹²³I and ¹⁸F.

The physical method for detecting the labelled isomer of the present invention may be selected from Position Emission Tomography (PET), Single Photon Imaging Computed Tomography (SPECT), Magnetic Resonance Spectroscopy (MRS), Magnetic Resonance Imaging (MRI), and Computed Axial X-ray Tomography (CAT), or combinations thereof.

### Methods of Preparation

The pyrimidine-2,4-diamine derivative of the invention may be prepared by conventional methods for chemical synthesis, e.g. those described in the working examples. The starting materials for the processes described in the present application are known or may readily be prepared by conventional methods from commercially available chemicals.

Also one compound of the invention can be converted to another compound of the invention using conventional methods.

The end products of the reactions described herein may be isolated by conventional techniques, e.g. by extraction, crystallisation, distillation, chromatography, etc.

### Biological Activity

The pyrimidine-2,4-diamine derivatives of the invention may be tested for their ability to modulate SK channels in vitro. Functional modulation can be determined by measuring the compound-induced change in SK current by the patch clamp technique as described in Strøbaek et al.: “Pharmacological characterization of small-conductance Ca²⁺-activated K channels expressed in HEK293 cells”, British Journal of Pharmacology (2000) 129, 991-999. From this type of measurements the potency of a given compound can be determined as e.g. Kᵢ or IC₅₀ values for blockers/inhibitors and EC₅₀ values for openers/activators. Similar data can be obtained from other patch clamp configurations and from channels expressed endogenously in various cell lines.

In one embodiment, the pyrimidine-2,4-diamine derivatives of the invention show selectivity for SK3 over SK1 and SK2. In a further embodiment, the compounds of the invention are positive SK channel modulators, such as positive SK3 channel modulators. In a still further embodiment, the compounds of the invention are negative modulators, such as negative SK3 channel modulators. In a special embodiment, the compounds of the invention are SK channel blockers, such as SK3 channel blockers.

Based on the activity observed in the patch clamp experiments, the compound of the invention is considered useful for the treatment, prevention or alleviation of a disease or a disorder or a condition of a mammal, including a human, which disease, disorder or condition is responsive to modulation of SK channels.

In a special embodiment, the pyrimidine-2,4-diamine derivatives of the invention are considered useful for the treatment, prevention or alleviation of absence seizures, agerelated memory loss, Alzheimer's disease, angina pectoris, arrhythmia, asthma, anxiety, ataxia, attention deficits, baldness, bipolar disorder, bladder hyperexcitability, bladder outflow obstruction, bladder spasms, brain tumors, cerebral ischaemia, chronic obstructive pulmonary disease, cancer, cardiovascular disorders, cognitive dysfunction, colitis, constipation, convulsions, coronary artery spasms, coronary hearth disease, cystic fibrosis, dementia, depression, diabetes type II, dysmenorrhoea, epilepsy, gastrointestinal dysfunction, gastroesophageal reflux disorder, gastrointestinal hypomotility disorders gastrointestinal motility insufficiency, hearing loss, hyperinsulinemia, hypertension, immune suppression, inflammatory bowel disease, inflammatory pain, intermittent claudication, irritable bowel syndrome, ischaemia, ischaemic hearth disease, learning deficiencies, male erectile dysfunction, manic depression, memory deficits, migraine, mood disorders, motor neuron diseases, myokymia, myotonic dystrophy, myotonic muscle dystrophia, narcolepsy, neuropathic pain, pain, Parkinson's disease, polycystic kidney disease, postoperative ileus, premature labour, psychosis, psychotic disorders, renal disorders, Reynaud's disease, rhinorrhoea, secretory diarrhoea, seizures, Sjorgren's syndrome, sleep apnea, spasticity, sleeping disorders, stroke, traumatic brain injury, trigeminal neuralgia, urinary incontinence, urinogenital disorders, vascular spasms, vision loss, and xerostomia. In a more preferred embodiment the compounds of the invention are considered useful for the treatment, prevention or alleviation of depression, pseudodementia, Ganser's syndrome, obsessive compulsive disorder, panic disorder, memory deficits, memory loss, attention deficit hyperactivity disorder, obesity, anxiety, eating disorder, Parkinson's disease, parkinsonism, dementia, dementia of ageing, senile dementia, acquired immunodeficiency syndrome dementia complex, memory dysfunction in ageing, social phobia, drug addiction, drug misuse, cocaine abuse, tobacco abuse, alcoholism, pain, migraine pain, bulimia, premenstrual syndrome, late luteal phase syndrome, post-traumatic syndrome, chronic fatigue syndrome, premature ejaculation, erectile difficulty, anorexia nervosa, sleep disorders, autism, mutism, trichotillomania, narcolepsy, Gilles de la Tourettes disease, inflammatory bowel disease or irritable bowel syndrome.

In another more preferred embodiment the pyrimidine-2,4-diamine derivatives of the invention are considered useful for the treatment, prevention or alleviation of depression, pseudodementia, Ganser's syndrome, obsessive compulsive disorders, panic disorders, memory deficits, attention deficit hyperactivity disorder, obesity, anxiety, an eating disorder or Parkinson's disease.

In a third more preferred embodiment, the pyrimidine-2,4-diamine derivatives of the invention are considered useful for the treatment, prevention or alleviation of a respiratory disease, urinary incontinence, erectile dysfunction, anxiety, epilepsy, psychosis, schizophrenia, bipolar disorder, depression, amyotrophic lateral sclerosis (ALS), Parkinson's disease or pain.

In a fourth more preferred embodiment, the pyrimidine-2,4-diamine derivatives of the invention are considered useful for the treatment, prevention or alleviation of psychosis, schizophrenia, bipolar disorder, depression, epilepsy, Parkinson's disease or pain.

In a fifth more preferred embodiment, the pyrimidine-2,4-diamine derivatives of the invention are considered useful for the treatment, prevention or alleviation of pain, mild or moderate or severe pain, pain of acute, chronic or recurrent character, pain caused by migraine, postoperative pain, phantom limb pain, inflammatory pain, neuropathic pain, chronic headache, central pain, pain related to diabetic neuropathy, to post therapeutic neuralgia, or to peripheral nerve injury.

In a most preferred embodiment, the pyrimidine-2,4-diamine derivatives of the invention are considered useful for the treatment, prevention or alleviation of schizophrenia, depression or Parkinson's disease.

It is at present contemplated that a suitable dosage of the active pharmaceutical ingredient (API) is within the range of from about 0.1 to about 1000 mg API per day, more preferred of from about 10 to about 500 mg API per day, most preferred of from about 30 to about 100 mg API per day, dependent, however, upon the exact mode of administration, the form in which it is administered, the indication considered, the subject and in particular the body weight of the subject involved, and further the preference and experience of the physician or veterinarian in charge.

Preferred pyrimidine-2,4-diamine derivatives of the invention show a biological activity in the sub-micromolar and micromolar range, i.e. of from below 1 to about 100 μM.

### Pharmaceutical Compositions

In another aspect the invention provides novel pharmaceutical compositions comprising a therapeutically effective amount of the pyrimidine-2,4-diamine derivative of the invention.

While a pyrimidine-2,4-diamine derivative of the invention for use in therapy may be administered in the form of the raw chemical compound, it is preferred to introduce the active ingredient, optionally in the form of a physiologically acceptable salt, in a pharmaceutical composition together with one or more adjuvants, excipients, carriers, buffers, diluents, and/or other customary pharmaceutical auxiliaries.

In a preferred embodiment, the invention provides pharmaceutical compositions comprising the pyrimidine-2,4-diamine derivative of the invention, or a pharmaceutically acceptable salt or derivative thereof, together with one or more pharmaceutically acceptable carriers, and, optionally, other therapeutic and/or prophylactic ingredients, known and used in the art. The carrier(s) must be “acceptable” in the sense of being compatible with the other ingredients of the formulation and not harmful to the recipient thereof.

The pharmaceutical composition of the invention may be administered by any convenient route, which suits the desired therapy. Preferred routes of administration include oral administration, in particular in tablet, in capsule, in dragé, in powder, or in liquid form, and parenteral administration, in particular cutaneous, subcutaneous, intramuscular, or intravenous injection. The pharmaceutical composition of the invention can be prepared by any skilled person by use of standard methods and conventional techniques appropriate to the desired formulation. When desired, compositions adapted to give sustained release of the active ingredient may be employed.

Further details on techniques for formulation and administration may be found in the latest edition of Remington's Pharmaceutical Sciences (Maack Publishing Co., Easton, Pa.).

The actual dosage depends on the nature and severity of the disease being treated, and is within the discretion of the physician, and may be varied by titration of the dosage to the particular circumstances of this invention to produce the desired therapeutic effect. However, it is presently contemplated that pharmaceutical compositions containing of from about 0.1 to about 500 mg of active ingredient per individual dose, preferably of from about 1 to about 100 mg, most preferred of from about 1 to about 10 mg, are suitable for therapeutic treatments.

The active ingredient may be administered in one or several doses per day. A satisfactory result can, in certain instances, be obtained at a dosage as low as 0.1 μg/kg i.v. and 1 μg/kg p.o. The upper limit of the dosage range is presently considered to be about 10 mg/kg i.v. and 100 mg/kg p.o. Preferred ranges are from about 0.1 μg/kg to about 10 mg/kg/day i.v., and from about 1 μg/kg to about 100 mg/kg/day p.o.

### Methods of Therapy

In another aspect the invention provides a method for the treatment, prevention or alleviation of a disease or a disorder or a condition of a living animal body, including a human, which disease, disorder or condition is responsive to modulation of SK channels, and which method comprises administering to such a living animal body, including a human, in need thereof an effective amount of a pyrimidine-2,4-diamine derivative of the invention.

It is at present contemplated that suitable dosage ranges are 0.1 to 1000 milligrams daily, 10-500 milligrams daily, and especially 30-100 milligrams daily, dependent as usual upon the exact mode of administration, form in which administered, the indication toward which the administration is directed, the subject involved and the body weight of the subject involved, and further the preference and experience of the physician or veterinarian in charge.

### EXAMPLES

The invention is further illustrated with reference to the following examples, which are not intended to be in any way limiting to the scope of the invention as claimed.

General: The procedures represent generic procedures used to prepare the pyrimidine-2,4-diamine derivatives of the invention. Abbreviations used are as follows:
Me: methyl
mp: melting point
MW: microwave
rt: room temperature

### Procedure A

2,4-Dichloropyrimidine and two equivalents of the required amine were suspended in acetonitrile in a closed vial and heated to 150-200° C. for 15-45 min by use of microwave (MW) irradiation. After cooling to room temperature the precipitated solid was filtered off and purified by column chromatography or preparative LCMS to give the desired product as the free base. Alternatively, the product was isolated as an HCl salt by precipitation from a mixture of HCl in water/acetonitrile.

An example of Procedure A, the preparation of N²,N⁴-bis(3,4-difluorobenzyl)pyrimidine-2,4-diamine, is shown in Scheme 1.

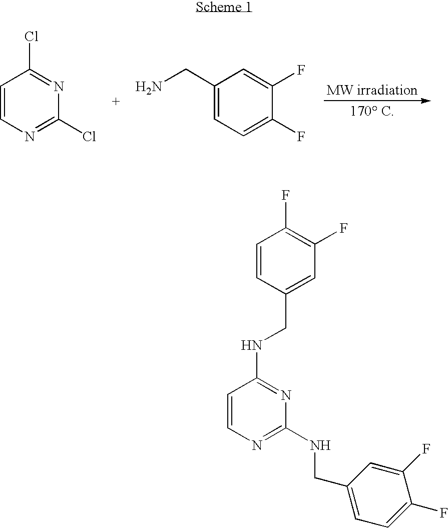

### Example 1

### N²,N⁴-Bis(3,4-difluorobenzyl)pyrimidine-2,4-diamine

The title compound was prepared from 2,4-dichloropyrimidine and 3,4-difluorobenzylamine by Procedure A. The product was isolated by filtration and subsequent purification by column chromatography to give the title compound as a yellowish oil. MS (ES⁺) m/z 363 ([M+1]⁺, 100). ¹H NMR (DMSO-d6) δ 4.31 (s, 2H), 4.39 (s, 2H), 5.76 (d, 1H), 6.92-7.54 (m, 8H), 7.65 (d, 1H).

### Example 2

### N²,N⁴-Bis(3,4-difluorobenzyl)-6-methylpyrimidine-2,4-diamine

The title compound was prepared from 2,4-dichloro-6-methylpyrimidine and 3,4-difluorobenzylamine by Procedure A. The product was isolated by filtration and subsequent purification by preparative LCMS to give the title compound as a yellowish oil. MS (ES⁺) m/z 377 ([M+1]⁺, 100). ¹H NMR (DMSO-d6) δ 2.01 (s, 3H), 4.32 (s, 2H), 4.37 (s, 2H), 5.65 (s, 1H), 6.92-7.42 (m, 8H).

### Example 3

### N²,N⁴-Bis[3-(trifluoromethyl)benzyl]pyrimidine-2,4-diamine

The title compound was prepared from 2,4-dichloropyrimidine and 3-(trifluoromethyl)benzylamine by Procedure A. The product was isolated by preparative LCMS and subsequent precipitation as an HCl salt (white solid, mp 172° C.). MS (ES⁺) m/z 427 ([M+1]⁺, 100).

### Example 4

### N²,N⁴-Bis(3,4-dichlorobenzyl)pyrimidine-2,4-diamine

The title compound was prepared from 2,4-dichloropyrimidine and 3,4-dichlorobenzylamine by Procedure A. The product was isolated by preparative LCMS and subsequent precipitation as an HCl salt (white solid, mp 167-170° C.). MS (ES⁺) m/z 429 ([M+1]⁺, 100).

### Example 5

### N²,N⁴-Bis[1-(4-fluorophenyl)ethyl]pyrimidine-2,4-diamine

The title compound was prepared from 2,4-dichloropyrimidine and 1-(4-fluorophenyl)ethylamine by Procedure A. The product was isolated by preparative LCMS and subsequent precipitation as an HCl salt (off-white solid, mp 151-154° C.). MS (ES⁺) m/z 355 ([M+1]⁺, 100).

### Example 6

### N²,N⁴-Bis[4-fluoro-3-(trifluoromethyl)benzyl]pyrimidine-2,4-diamine

The title compound was prepared from 2,4-dichloropyrimidine and 4-fluoro-3-(trifluoromethyl)benzylamine by Procedure A. The product was isolated by preparative LCMS to give the title compound as a colourless oil. MS (ES⁺) m/z 463 ([M+1]⁺, 100). ¹H NMR (DMSO-d6) δ 4.39 (s, 2H), 4.45 (s, 2H), 5.80 (d, 1H), 7.05-7.75 (m, 9H).

### Example 7

### N²,N⁴-Bis(4-chlorobenzyl)pyrimidine-2,4-diamine

The title compound was prepared from 2,4-dichloropyrimidine and 4-chlorobenzylamine by Procedure A. The product was isolated by preparative LCMS and subsequent precipitation as an HCl salt (white solid, mp 213-216° C.). MS (ES⁺) m/z 359 (M⁺, 100).

### Procedure B

2,4-Dichloropyrimidine dissolved in acetonitrile was cooled on ice and added 1.2 eq of the required amine (Amine A, Scheme 2). After stirring overnight at rt, water was added to the reaction mixture and it was extracted with EtOAc. The combined organic phases were dried (MgSO₄), filtered and concentrated in vacuo to give the crude 4-arylalkylamino-2-chloropyrimidine which was isolated by column chromatography. Subsequently, this intermediate was dissolved in acetonitrile, added 1.2 eq of Amine B (Scheme 2) and the reaction mixture heated to 150-200° C. for 15-80 min by use of MW irradiation.

Alternatively, the crude reaction mixture after step 1 was used without isolation of the 4-arylalkylamino-2-chloropyrimidine, added Amine B and heated in the MW oven.

After step 2, the crude product was isolated by aqueous work-up, as described above, or by filtration from the reaction mixture. The crude product could subsequently be purified by column chromatography or preparative LCMS to yield the desired 2,4-bis(arylalkylamino)pyrimidine as the free base. Alternatively, this product was isolated as an HCl salt upon filtration of the reaction mixture and recrystallization.

An example of Procedure B, the preparation of 2-{benzyl[4-(4-chlorobenzylamino)-pyrimidin-2-yl]amino}ethanol, is shown in Scheme 2.

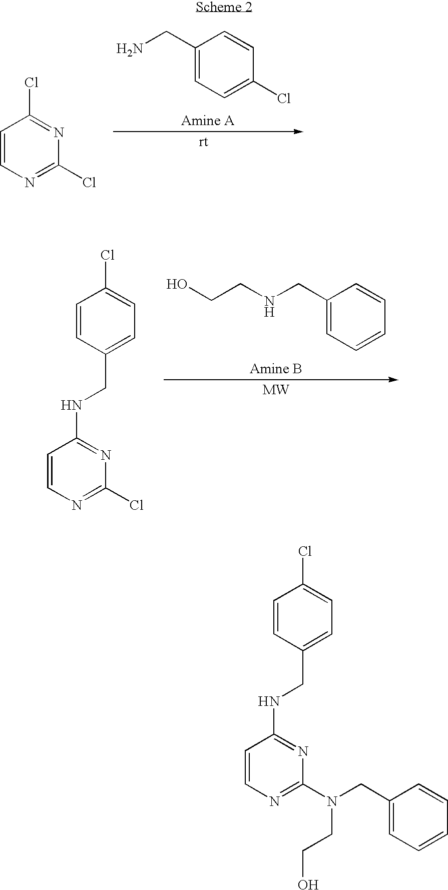

### Example 8

### N²,N⁴-Bis(4-chlorobenzyl)-N²,N⁴-dimethyl-pyrimidine-2,4-diamine

The title compound was prepared from 2,4-dichloropyrimidine and N-(4-chlorobenzyl)-N-methylamine by Procedure A. The reaction mixture was added saturated aqueous NaHCO₃ and extracted with EtOAc. The combined organic phases were dried (MgSO₄), filtered and concentrated in vacuo. The crude product was purified by column chromatography to give the title compound as a yellowish oil. MS (ES⁺) m/z 387 ([M+1]⁺, 100). ¹H NMR (DMSO-d6) δ 2.94 (s, 3H), 3.00 (s, 3H), 4.66 (s, 2H), 4.72 (s, 2H), 5.96 (d, 1H), 7.05-7.35 (m, 8H), 7.85 (d, 1H).

### Example 9

### 2-{Benzyl[4-(4-chlorobenzylamino)pyrimidin-2-yl]amino}ethanol

The title compound was prepared in two steps by Procedure B from 2,4-dichloropyrimidine, 4-chlorobenzylamine and N-benzylethanolamine. The reaction mixture was added water and extracted with EtOAc. The combined organic phases were dried (MgSO₄), filtered and concentrated in vacuo. The crude product was purified by column chromatography to give the title compound as a yellowish oil. MS (ES⁺) m/z 369 ([M+1]⁺, 100); HR-MS: 369.148400 ([M+1]⁺, C₂₀H₂₂ClN₄O; calc. 369.148214).

### Example 10

### N²,N⁴-Bis[2-(4-fluorophenyl)ethyl]pyrimidine-2,4-diamine

The title compound was prepared from 2,4-dichloropyrimidine and 4-fluorobenzylamine by Procedure A. The product was isolated by column chromatography and subsequent precipitation as an HCl salt (white solid, mp 177.4-179.4° C.). MS (ES⁺) m/z 355 ([M+1]⁺, 100).

### Example 11

### N²,N⁴-Bis[2-(4-chlorophenyl)ethyl]pyrimidine-2,4-diamine

The title compound was prepared from 2,4-dichloropyrimidine and 4-chlorobenzylamine by Procedure A. The product was isolated by column chromatography and sub-sequent precipitation as an HCl salt (white solid, mp 201-204° C.). MS (ES⁺) m/z 388 ([M+1]⁺, 100).

### Example 12

### N²,N⁴-Di-(R)-1,2,3,4-tetrahydronaphthalen-1-yl-pyrimidine-2,4-diamine

The title compound was prepared from 2,4-dichloropyrimidine and (R)-1,2,3,4-tetrahydro-1-naphthylamine by Procedure A. The product was isolated by preparative LCMS to give the title compound as a colourless oil. MS (ES⁺) m/z 371 ([M+1]⁺, 100). ¹NMR (CDCl₃) δ 1.69-2.05 (m, 8H), 2.62-2.80 (m, 4H), 5.16 (m, 2H), 5.67 (m, 1H), 6.97-7.13 (m, 6H), 7.23-7.35 (m, 2H), 7.62-7.80 (m, 1H).

### Example 13

### N²,N⁴-Dibenzyl-N²,N⁴-bis(2-dimethylaminoethyl)pyrimidine-2,4-diamine

The title compound was prepared from 2,4-dichloropyrimidine and N′-benzyl-N,N-dimethylethylenediamine by Procedure A. The product was isolated by preparative LCMS to give the title compound as a colourless oil. MS (ES⁺) m/z 433 ([M+1]⁺, 100). ¹NMR (CDCl₃) δ 2.11 (br s, 12H), 2.30-2.48 (4H), 2.97-3.55 (m, 4H), 4.65 (br s, 2H), 4.80 (s, 2H), 5.75 (m, 1H), 7.00-7.37 (m, 10H), 7.85 (m, 1H).

### Example 14

### N²,N⁴-Bis(4-dimethylaminobenzyl)pyrimidine-2,4-diamine

The title compound was prepared from 2,4-dichloropyrimidine and 4-(dimethylamino)benzylamine by Procedure A. The product was isolated by preparative LCMS to give the title compound as a colourless oil. MS (ES⁺) m/z 377 ([M+1]⁺, 100). ¹NMR (DMSO-d6) δ 2.86 (s, 12H), 4.29 (s, 2H), 4.31 (s, 2H), 5.70 (m, 1H), 6.60-6.70 (m, 4H), 7.07-7.15 (m, 4H), 7.60 (m, 1H).

1-20. (canceled)

21. A pyrimidine-2,4-diamine derivative of Formula I: 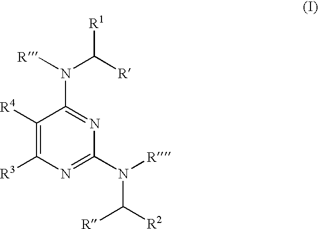
any of its stereoisomers or any mixture of its stereoisomers, or a pharmaceutically acceptable salt thereof, wherein
R¹ represents —(CH₂)ᵥ—R⁵; wherein
v is 0 or 1; and
R⁵ represents an aryl group, which aryl group is optionally substituted with one or more substituents independently selected from the group consisting of halo, trifluoromethyl, trifluoromethoxy, cyano, alkyl, amino or N,N-dialkyl-amino; and
R′ and R′″, independent of each other, represent hydrogen or R^{e}-alkyl; or
R′ together with R′″ form —(CH₂)ₚ—, wherein p is 3, 4 or 5; or
R′ forms a —(CH₂)_{q}— bridge to an ortho position of the aryl group of R¹, wherein q is 2, 3 or 4; and R′″ represents hydrogen or R^{e}-alkyl;
wherein R^{e} represents hydrogen, hydroxyl, cyano, amino or N,N-dialkyl-amino; and
R² represents —(CH₂)_{w}—R⁶; wherein
w is 0 or 1; and
R⁶ represents an aryl group, which aryl group is optionally substituted with one or more substituents independently selected from the group consisting of halo, trifluoromethyl, trifluoromethoxy, cyano, alkyl, amino or N,N-dialkyl-amino; and
R″ and R″″ independent of each other represent hydrogen or R^{f}-alkyl; or
R″ together with R″″ form —(CH₂)ₛ—, wherein s is 3, 4 or 5; or
R″ forms a —(CH₂)ₜ— bridge to an ortho position of the aryl group of R², wherein t is 2, 3 or 4; and R″″ represents hydrogen or R^{f}-alkyl;
wherein R^{f} represents hydrogen, hydroxyl, cyano, amino or N,N-dialkyl-amino; and
R³ and R⁴ independent of each other are selected from the group consisting of hydrogen, halo, trifluoromethyl, trifluoromethoxy, cyano, alkyl and alkoxy.

22. The pyrimidine-2,4-diamine derivative of claim 21, or a pharmaceutically acceptable salt thereof, wherein
R¹ represents —(CH₂)ᵥ—R⁵; wherein
v is 0 or 1; and
R⁵ represents an aryl group, which aryl group is optionally substituted with one or more substituents independently selected from the group consisting of halo, trifluoromethyl, trifluoromethoxy, cyano, alkyl, amino or N,N-dialkyl-amino.

23. The pyrimidine-2,4-diamine derivative of claim 21, or a pharmaceutically acceptable salt thereof, wherein
R′ and R′″, independent of each other, represent hydrogen or R^{e}-alkyl; or
R′ together with R′″ form —(CH₂)ₚ—, wherein p is 3, 4 or 5; or
R′ forms a —(CH₂)_{q}— bridge to an ortho position of the aryl group of R¹, wherein q is 2, 3 or 4; and R′″ represents hydrogen or R^{e}-alkyl;
wherein R^{e} represents hydrogen, hydroxyl, cyano, amino or N,N-dialkyl-amino.

24. The pyrimidine-2,4-diamine derivative of claim 21, or a pharmaceutically acceptable salt thereof, wherein
R² represents —(CH₂)_{w}—R⁶; wherein
w is 0 or 1; and
R⁶ represents an aryl group, which aryl group is optionally substituted with one or more substituents independently selected from the group consisting of halo, trifluoromethyl, trifluoromethoxy, cyano, alkyl, amino or N,N-dialkyl-amino.

25. The pyrimidine-2,4-diamine derivative of claim 21, or a pharmaceutically acceptable salt thereof, wherein
R″ and R″″ independent of each other represent hydrogen or R^{f}-alkyl; or
R″ together with R″″ form —(CH₂)ₛ—, wherein s is 3, 4 or 5; or
R″ forms a —(CH₂)ₜ— bridge to an ortho position of the aryl group of R², wherein t is 2, 3 or 4; and R″″ represents hydrogen or R^{f}-alkyl;
wherein R^{f} represents hydrogen, hydroxyl, cyano, amino or N,N-dialkyl-amino.

26. The pyrimidine-2,4-diamine derivative of claim 21, or a pharmaceutically acceptable salt thereof, wherein
R³ and R⁴ independent of each other are selected from the group consisting of hydrogen, halo, trifluoromethyl, trifluoromethoxy, cyano, alkyl and alkoxy.

27. The pyrimidine-2,4-diamine derivative of claim 21, or a pharmaceutically acceptable salt thereof, wherein
R′, R″, R′″ and R″″ independent of each other are hydrogen or alkyl;
R¹ represents an aryl group;
which aryl group is optionally substituted with one or more substituents independently selected from the group consisting of halo, trifluoromethyl, trifluoromethoxy, cyano and alkyl;
R² represents an aryl group;
which aryl group is optionally substituted with one or more substituents independently selected from the group consisting of halo, trifluoromethyl, trifluoromethoxy, cyano and alkyl
R³ and R⁴ independent of each other are selected from the group consisting of hydrogen, halo, trifluoromethyl, trifluoromethoxy, cyano, alkyl and alkoxy.

28. The pyrimidine-2,4-diamine derivative of claim 21, or a pharmaceutically acceptable salt thereof, wherein
R¹ represents 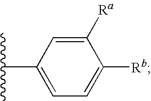
wherein R^{a} and R^{b} independent of each other are selected from the group consisting of hydrogen, halo, trifluoromethyl, trifluoromethoxy, cyano and alkyl.

29. The pyrimidine-2,4-diamine derivative of claim 21, or a pharmaceutically acceptable salt thereof, wherein
R² represents 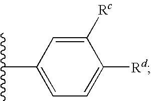
wherein R^{c} and R^{d} independent of each other are selected from the group consisting of hydrogen, halo, trifluoromethyl, trifluoromethoxy, cyano and alkyl.

30. The pyrimidine-2,4-diamine derivative of claim 21, or a pharmaceutically acceptable salt thereof, wherein
R³ represents hydrogen or alkyl.

31. The pyrimidine-2,4-diamine derivative of claim 21, or a pharmaceutically acceptable salt thereof, wherein
R⁴ represents hydrogen or alkyl.

32. The pyrimidine-2,4-diamine derivative of claim 21, or a pharmaceutically acceptable salt thereof, wherein
R′ represents hydrogen or alkyl.

33. The pyrimidine-2,4-diamine derivative of claim 21, or a pharmaceutically acceptable salt thereof, wherein
R″ represents hydrogen or alkyl.

34. The pyrimidine-2,4-diamine derivative of claim 21, wherein
R′″ and R″″ represent hydrogen.

35. The pyrimidine-2,4-diamine derivative of claim 21, which is
N²,N⁴-Bis(3,4-difluorobenzyl)pyrimidine-2,4-diamine;
N²,N⁴-Bis(3,4-difluorobenzyl)-6-methylpyrimidine-2,4-diamine;
N²,N⁴-Bis[3-(trifluoromethyl)benzyl]pyrimidine-2,4-diamine;
N²,N⁴-Bis(3,4-dichlorobenzyl)pyrimidine-2,4-diamine;
N²,N⁴-Bis[1-(4-fluorophenyl)ethyl]pyrimidine-2,4-diamine;
N²,N⁴-Bis[4-fluoro-3-(trifluoromethyl)benzyl]pyrimidine-2,4-diamine;
N²,N⁴-Bis(4-chlorobenzyl)pyrimidine-2,4-diamine;
N²,N⁴-Bis(4-chlorobenzyl)-N²,N⁴-dimethyl-pyrimidine-2,4-diamine;
2-{Benzyl[4-(4-chlorobenzylamino)pyrimidin-2-yl]amino}ethanol;
N²,N⁴-Bis[2-(4-fluorophenyl)ethyl]pyrimidine-2,4-diamine;
N²,N⁴-Bis[2-(4-chlorophenyl)ethyl]pyrimidine-2,4-diamine;
N²,N⁴-Di-(R)-1,2,3,4-tetrahydronaphthalen-1-yl-pyrimidine-2,4-diamine;
N²,N⁴-Dibenzyl-N²,N⁴-bis(2-dimethylaminoethyl)pyrimidine-2,4-diamine; or
N²,N⁴-Bis(4-dimethylaminobenzyl)pyrimidine-2,4-diamine;
or a pharmaceutically acceptable salt thereof.

36. A pharmaceutical composition, comprising a therapeutically effective amount of the pyrimidine-2,4-diamine derivative of claim 21, or any of its stereoisomers or any mixture of its stereoisomers, or a pharmaceutically acceptable salt thereof, together with at least one pharmaceutically acceptable carrier, excipient or diluent.

37. A method for treatment, prevention or alleviation of a disease or a disorder or a condition of a living animal body, including a human, which disorder, disease or condition is responsive to modulation of SK channels, which method comprises the step of administering to such a living animal body in need thereof a therapeutically effective amount of the pyrimidine-2,4-diamine derivative according to claim 1, or any of its stereoisomers or any mixture of its stereoisomers, or a pharmaceutically acceptable salt thereof.

38. The method according to claim 37, wherein the disease, disorder or condition responsive to modulation of SK channels is: absence seizures, agerelated memory loss, Alzheimer's disease, angina pectoris, arrhythmia, asthma, anxiety, ataxia, attention deficits, baldness, bipolar disorder, bladder hyperexcitability, bladder outflow obstruction, bladder spasms, brain tumors, cerebral ischaemia, chronic obstructive pulmonary disease, cancer, cardiovascular disorders, cognitive dysfunction, colitis, constipation, convulsions, coronary artery spasms, coronary hearth disease, cystic fibrosis, dementia, depression, diabetes type II, dysmenorrhoea, epilepsy, gastrointestinal dysfunction, gastroesophageal reflux disorder, gastrointestinal hypomotility disorders gastrointestinal motility insufficiency, hearing loss, hyperinsulinemia, hypertension, immune suppression, inflammatory bowel disease, inflammatory pain, intermittent claudication, irritable bowel syndrome, ischaemia, ischaemic hearth disease, learning deficiencies, male erectile dysfunction, manic depression, memory deficits, migraine, mood disorders, motor neuron diseases, myokymia, myotonic dystrophy, myotonic muscle dystrophia, narcolepsy, neuropathic pain, pain, Parkinson's disease, polycystic kidney disease, postoperative ileus, premature labour, psychosis, psychotic disorders, renal disorders, Reynaud's disease, rhinorrhoea, secretory diarrhoea, seizures, Sjorgren's syndrome, sleep apnea, spasticity, sleeping disorders, stroke, traumatic brain injury, trigeminal neuralgia, urinary incontinence, urinogenital disorders, vascular spasms, vision loss, or xerostomia.
